(19) United States
(12) Patent Application Publication
(10) Pub. No.: US 2004/0087797 A1
(43) Pub. Date: May 6, 2004

(54) Dihydro-furan-2-one derivatives, their intermediates and methods of manufacture

(75) Inventor: Zhengong B. Li, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/687,153

(22) Filed: Oct. 16, 2003

Related U.S. Application Data
(60) Provisional application No. 60/422,737, filed on Oct. 31, 2002.

(51) Int. Cl. C07D 307/26; C 07D 4 5/02

(52) U.S. Cl. 546/284.4; 549/321

(57) ABSTRACT

This invention relates to dihydro-furan-2-one derivatives, their intermediates and methods of manufacture. As such, the present invention includes methods of making a compound of the formula (V-1)

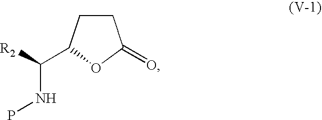

wherein R₂ and P are herein defined. The present invention also relates to the compounds used in such processes.

### PRIORITY CLAIM

The present application claims priority to U.S. Patent Application Serial No. 60/422,737, filed Oct. 31, 2002, which is incorporated herein in its entirety.

### FIELD OF THE INVENTION

This invention relates to dihydro-furan-2-one derivatives, their intermediates, and methods of manufacture.

### BACKGROUND

Dihydroxyhexanoic acid derivatives are potent and selective inhibitors of MIP-1α, binding to the receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokine shown to interact with CCR1 (e.g., RANTES and MCP-3)) induced chemotaxis of THP-1 cells and human leukocytes and are potentially useful for the treatment or prevention of various diseases and conditions

Dihydroxyhexanoic acid derivatives and their methods of manufacture are disclosed in commonly assigned U.S. Pat. No. 6,403,587B1, filed Feb. 5, 1998, U.S. patent application Ser. No. 09/403,218, filed Jan. 18, 1999, U.S. patent application Ser. No. 09/774,871, filed Feb. 4, 2000, PCT Publication No. WO98/38167, PCT Publication No. WO99/40061, and PCT Publication No. WO01/57023, all of which are incorporated herein by reference in their entireties for all purposes.

### SUMMARY OF THE INVENTION

As embodied and broadly described herein, this invention, in one aspect, relates to methods of making a compound of the formula (V-1):

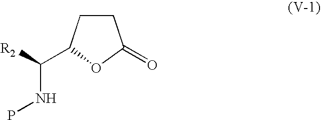

wherein:

P is a protecting group;

R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and

m is 0, 1, 2, 3, or 4;

wherein the method comprises:

a) reacting a compound of the formula (VIh-1)

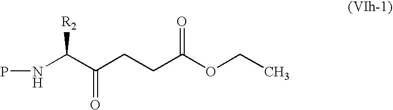

with a reducing agent, and

b) cyclizing the compound so formed with heat and an acid catalyst.

In one embodiment, the compound of the formula (VIh-1) is formed by reacting a compound of the formula (VIIa-1)

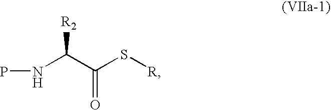

wherein R is a (C₁-C₂₀)alkyl, (C₃-C₁₀)cycloalkyl, aryl, or (C₂-C₉)heteroaryl with an organozinc compound in the presence of a palladium catalyst and a racemization scavenger.

In another embondiment, the compound of the formula (VIIa-1) is formed by reacting a compound of the formula (VIII-1)

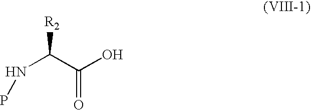

with a compound of the formula HS-R.

A second aspect of the present invention relates to methods of making a compound of the formula (V-1):

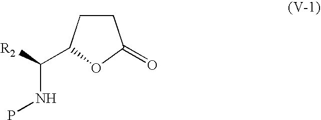

wherein:

P is a protecting group;

R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(CJ-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and

m is 0, 1, 2, 3, or 4;

wherein the method comprises:

a) reacting a compound of the formula (VIII-1)

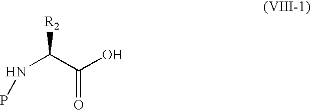

with a compound of the formula HS-R, wherein R is a (C₁-C₂₀)alkyl, (C₃-C₁₀)cycloalkyl, aryl, or (C₂-C₉)heteroaryl, to form a compound of the formula (VIIa-1)

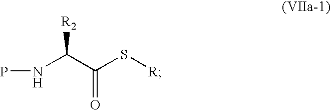

b) reacting the compound so formed with an organozinc compound in the presence of a palladium catalyst and a racemization scavenger to form a compound of the formula (VIh-1)

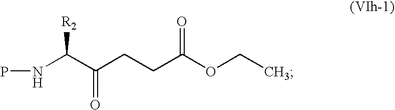

c) reacting the compound so formed with a reducing agent; and

d) cyclizing the compound so formed with heat and an acid catalyst.

In one embodiment of the present methods, the organozinc compound has the formula X—Zn—(C₁-C₆)alkyl-(C═O)—O—R, more preferably the formula,

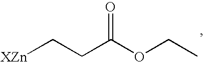

wherein X is a halogen and R is a (C₁-C₂₀)alkyl, (C₃-C₁₀)cycloalkyl, aryl, or (C₂-C₉)heteroaryl.

In another embodiment of the present methods, the organozinc compound has the formula

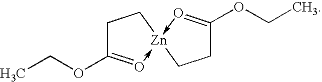

In yet another embodiment, the racemization scavenger is a carboxylic acid anhydride or a carboxylic acid ester. In another embodiment, the racemization scavenger has the formula aryl-(C═O)—O—(C═O)-aryl, R—(C═O)—O-aryl, or aryl-(C═O)—O-aryl, such as phthalic anhydride, 4-nitrophenyl acetate, or 4-fluorophenyl acetate.

In a third aspect, the present invention relates to compounds of the formula

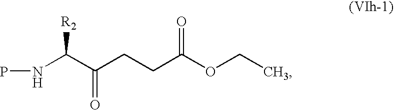

wherein:

P is a protecting group;

R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and

m is 0, 1, 2, 3, or 4.

In a fourth aspect, the present invention relates to compounds of the formula (VIIa-1)

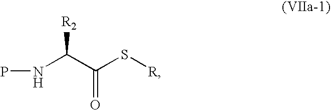

wherein:

P is a protecting group;

R is a (C₁-C₂₀)alkyl, (C₃-C₁₀)cycloalkyl, aryl, or (C₂-C₉)heteroaryl;

R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(0=C)—, (C₁-C₆)alkyl(0=C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and

m is 0, 1, 2, 3, or 4.

In certain embodiments of the aforementioned methods and compounds P is carbobenzyloxy, t-butoxy carbonyl or 9-fluorenyl-methylenoxycarbonyl, or P is t-butoxy carbonyl; R₂ is phenyl-(CH₂)ₘ— or (C₂-C₉)heteroaryl-(CH₂)ₘ—, wherein each of said phenyl or (C₂-C₉)heteroaryl moieties may be optionally substituted with one, two, or three substituents independently selected from the group consisting of hydrogen, halogen, CN, (C₁-C₆)alkyl, or hydroxy, or R₂ is 3-fluoro-benzyl; and R is a (C₁-C₆)alkyl, aryl, or (C₂-C₉)heteroaryl.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds and methods are disclosed and described, it is to be understood that this invention is not limited to specific synthetic methods of making that may of course vary. It is also to be understood that the terminology used herein is for the purpose of describing particular embodiments only and is not intended to be limiting.

In this specification and in the claims that follow, reference will be made to a number of terms that shall be defined to have the following meanings:

Unless otherwise indicated, the alkyl and alkenyl groups referred to herein, as well as the alkyl moieties of other groups referred to herein (e.g., alkoxy), may be linear or branched, and they may also be cyclic (e.g., cyclopropyl, cyclobutyl, cyclopentyl, cyclohexyl or cycloheptyl) or be linear or branched and contain cyclic moieties. Such alkyl and alkoxy groups may be substituted with one, two or three halogen and/or hydroxy atoms, preferably fluorine atoms.

Unless otherwise indicated, “halogen” and “halide” includes fluorine, chlorine, bromine, and iodine.

“(C₃-C₁₀)cycloalkyl” when used herein refers to cycloalkyl groups containing zero to two levels of unsaturation such as cyclopropyl, cyclobutyl, cyclopentyl, cyclopentenyl, cyclohexyl, cyclohexenyl, 1,3-cyclohexadiene, cycloheptyl, cycloheptenyl, bicyclo[3.2.1]octane, norbornanyl, and the like.

“(C₂-C₉)heterocycloalkyl” when used herein refers to pyrrolidinyl, tetrahydrofuranyl, dihydrofuranyl, tetrahydropyranyl, pyranyl, thiopyranyl, aziridinyl, oxiranyl, methylenedioxyl, chromenyl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, chromanyl, and the like. One of ordinary skill in the art will understand that the connection of said (C₂-C₉)heterocycloalkyl rings is through a carbon or a sp³ hybridized nitrogen heteroatom.

“(C₂-C₉)heteroaryl” when used herein refers to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5, 6, 7, 8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl, benzoxazinyl, and the like. One of ordinary skill in the art will understand that the connection of said (C₂-C₉)heterocycloalkyl rings is through a carbon atom or a sp³ hybridized nitrogen heteroatom.

“Aryl” when used herein refers to phenyl or naphthyl.

“Protected amine” and “protected amino” refers to an amine group with one of the hydrogen atoms replaced with a protecting group (P). Any suitable protecting group may be used for amine protection. Suitable protecting groups include carbobenzyloxy, t-butoxy carbonyl (BOC) or 9-fluorenyl-methylenoxy carbonyl.

By “pharmaceutically acceptable” is meant a material that is not biologically or otherwise undesirable, i.e., the material may be administered to an individual along with the selected compound without causing any undesirable biological effects or interacting in a deleterious manner with any of the other components of the pharmaceutical composition in which it is contained.

The term “subject” is meant an individual. Preferably, the subject is a mammal such as a primate, and more preferably, a human. Thus, the “subject” can include domesticated animals, livestock, and laboratory animals.

In general, “effective amount” or “effective dose” means the amount needed to achieve the desired result or results (treating or preventing the condition). One of ordinary skill in the art will recognize that the potency and, therefore, an “effective amount” can vary for the various compounds used in the invention. One skilled in the art can readily assess the potency of the compounds.

Unless otherwise noted, numerical values described and claimed herein are approximate. Variation within the values may be attributed to equipment calibration, equipment errors, purity of the materials, among other factors. Additionally, variation may be possible, while still obtaining the same result.

The compounds and processes of the present invention are useful in the manufacture of dihydroxyhexanoic acid derivatives. The present invention includes methods of making a compound of the formula (V-1) as shown in Scheme 1.

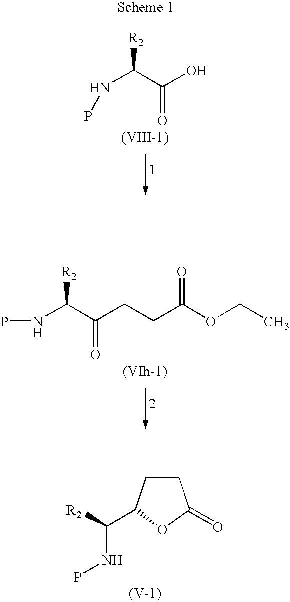

In Scheme 1 step 1, a compound of the formula (VIII-1) is reacted, in one embodiment, with a compound of the formula HS-R, wherein R is a (C₁-C₂₀)alkyl, (C₃-C₁₀)cycloalkyl, aryl, or (C₂-C₉)heteroaryl, to form a compound of the formula (VIIa-1). The compound of the formula (VIII-1) may be dissolved in an organic solvent, such as methylene chloride. To control the temperature of the heat of reaction, the temperature of the solution may be lowered to below room temperature, preferably below 0° C. Such a reaction produces a compound of the formula (VIIa-1)

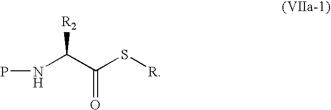

The compound of formula (VIIa-1) is reacted with an organozinc compound in the presence of a palladium catalyst and a racemization scavenger to form a compound of the formula (VIh-1). The organozinc compound preferably is an ethoxide with the formula X—Zn—(C₁-C₆)alkyl-(C═O)—O—R, more preferably the formula,

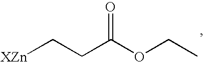

wherein X is a halogen and R is a (C₁-C₂₀)alkyl, (C₃-C₁₀)cycloalkyl, aryl, or (C₂-C₉)heteroaryl, such as 3-ethoxy-3-oxopropylzinc bromide. Exemplary racemization scavengers are carboxylic acid anhydrides or a carboxylic acid esters, such as those having the formula aryl-(C═O)—O—(C═O)-aryl, R—(C═O)—O-aryl, or aryl-(C═O)—O-aryl, which includes phthalic anhydride, 4-nitrophenyl acetate, and 4-fluorophenyl acetate. Any effective palladium catalyst may be used, such as dichlorobis-(triethylphosphine)palladium (II) under the temperature of about 0 to 65° C., preferably at 15-30° C.

In another embodiment, the compound of formula (VIII-1) is reacted with an organozinc compound of the formula

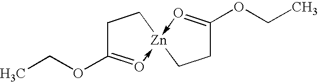

with a palladium catalyst and a racemization scavenger to directly form compounds of the formula (VIh-1). This organozinc compound may be made in situ by adding [1-(ethoxycycloprpopyl)oxy]trimethylsilane to Zn—X₂ in THF, wherein X is a halogen. Exemplary racemization scavengers are carboxylic acid anhydride sor a carboxylic acid esters, such as those having the formula aryl-(C═O)—O—(C═O)-aryl, R—(C═O)—O-aryl, or aryl-(C═O)—O-aryl, which includes phthalic anhydride, 4-nitrophenyl acetate, and 4-fluorophenyl acetate. Any effective palladium catalyst may be used, such as dichlorobis-(triethylphosphine)palladium (II) under the temperature of about 0 to 65° C., preferably at 15-30° C.

In step 2 of Scheme 1, the compound of formula (V-1) is formed by reacting the compound of formula (VIh-1) with a reducing agent and cyclizing the compound so formed with heat and an acid catalyst. In one embodiment, the compound (VIh-1) is reduced with N-selectride. The compound so formed may be cyclized with heat and an acid catalyst, such heating the compound in 10% acetic acid and toluene to provide the protected amine lactone (V-1).

The compounds of formula (V-1) are useful in the manufacture of dihydroxyhexanoic acid derivatives as shown in Schemes 2-4.

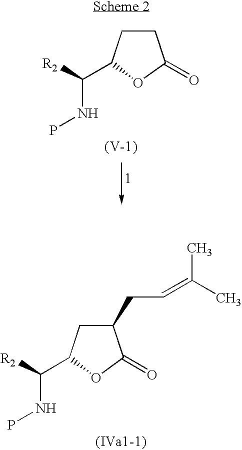

In step 1 of Scheme 2, the compound of the formula (IVa1-1) may be formed by reacting 4-halo-2-methyl-2-butene and a compound of the formula (V-1) in the presence of a base. Exemplary bases include lithium dialkyl amides such as lithium N-isopropyl-N-cyclohexylamide, lithium bis(trimethylsilyl)amide, lithium di-isopropylamide, and potassium hydride. Suitable solvents include aprotic polar solvents such as ethers (such as tetrahydrofuran, glyme or dioxane), benzene, or toluene, preferably tetrahydrofuran. The aforesaid reaction is conducted at a temperature from about −78° C. to about 0° C., preferably at about −78° C. In one embodiment, alkylation of the lactone (V-1) is accomplished by reacting the lactone (V-1) with lithium bis(trimethylsilyl)amide and dimethylallyl bromide in tetrahydrofuran at a temperature from about −78° C. to about −50° C. Reaction times range from several hours or if an additive such as dimethyl imidazolidinone is present, the reaction may be complete in minutes.

Compounds of formula (IVa1-1) may be used to produce compounds of the formula (Ia-1) according to Scheme 3:

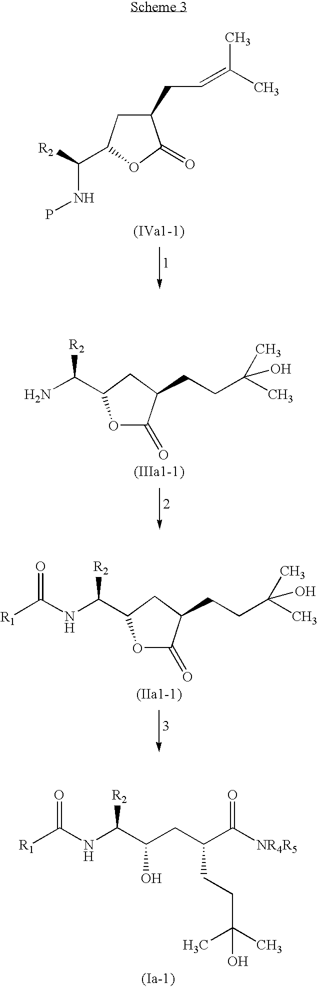

In step 1 of Scheme 3, a compound of the formula (IIIa1-1) is formed by reacting a compound of the formula (IVa1-1) with phosphoric acid. Preferably, this reaction occurs in any suitable solvent, such as non-alcoholic solvents. Two preferred solvents include tetrahydrofuran and dichloromethane. The reaction may take place at any suitable temperature, preferably from about −25° C. to about 120° C., more preferably from about 15° C. to about 40° C. Reaction time is dependent on temperature and batch size, amount other factors, but typically reaction time is from about 2 hours to about 14 hours.

Step 2 of Scheme 3 depicts coupling a compound IIIa1-1 with a compound having the formula R₁—CO—X to form a compound having the formula (IIa1-1). This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI), and diethylphosphorylcyanide. The coupling is conducted in an inert solvent, preferably an aprotic solvent, such as tetrahydrofuran, acetonitrile, dichloromethane, chloroform, or N,N-dimethylformamide. One preferred solvent is tetrahydrofuran. In one embodiment, quinoxaline acid is combined with CDI in anhydrous tetrahydrofuran and heated to provide the acyl imidazole. Compound IIIa1-1 is added to the acyl imidazole at room temperature to form the compound IIa1-1.

Step 3 of Scheme 3 includes reacting the compound of formula Ia1-1 with an amine having a formula NHR₄R₅ to form a compound of the formula (Ia-1). In one embodiment, the amine is ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa1-1 is dissolved in methanol which has been saturated with ammonia gas. In another embodiment, the compound IIa1-1 in methanol is treated with ammonium hydroxide in tetrahydrofuran at room temperature.

Scheme 4 represents an alternative method to form compounds of formula Ia-1 from compounds of formula IVa1-1.

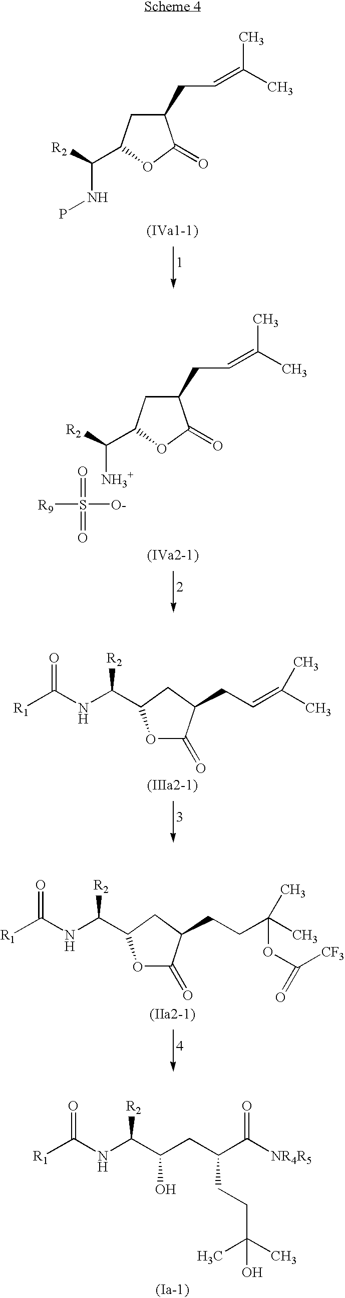

In step 1 of Scheme 4, a compound of the formula (IVa1-1) is reacted with a compound of the formula R₉—SO₂—OH to form a compound of the formula (IVa2-1). Any suitable acidic deprotection reaction may be performed. In one example, an excess of p-toluenesulfonic acid hydrate in ethyl acetate is introduced to the compound IVa1-1 at room temperature. Suitable solvents include ethyl acetate, alcohols, tetrahydrofuran, and mixtures thereof. The reaction may proceed at ambient or elevated temperatures. Typically, the reaction is substantially complete within two and twelve hours. The resulting compound IVa2-1 may be crystallized and separated from the reaction mixture, and may be further purified to remove impurities by recrystallization from hot ethyl acetate.

In step 2 of Scheme 4, the compound IVa2-1 may be coupled with a compound having the formula R₁—CO—X to form a compound of the formula (IIIa2-1). This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI)/dimethylaminopyridine (DMAP), and diethylphosphorylcyanide. The coupling is conducted in an inert solvent, preferably an aprotic solvent, such as acetonitrile, dichloromethane, chloroform, or N,N-dimethylformamide. One preferred solvent is methylene chloride. In one embodiment, quinoxaline acid is combined with methylene chloride, oxalyl chloride and a catalytic amount of N,N-dimethylformamide to form an acid chloride complex. The compound IVa2-1 is added to the acid chloride complex followed by triethylamine at a temperature from about 0° C. to about 25° C. to form the compound IIIa2-1.

Step 3 of Scheme 4 includes reacting a compound IIIa2-1 with trifluoroacetic acid to produce a compound of the formula (IIa2-1). In one embodiment, the hydration with trifluoroacetic acid occurs in methylene chloride solution at room temperature. The hydration may take several hours to complete at room temperature. A catalytic amount of sulfuric acid can be added to the reaction solution to increase the rate of reaction.

Step 4 of Scheme 4 includes reacting the compound of formula IIa2-1 with an amine having a formula NHR₄R₅ to form a compound of the formula (Ia-1). In one embodiment, the amine is ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa2-1 is dissolved in methanol which has been saturated with ammonia gas. In another embodiment, the compound IIa2-1 in methanol is treated with ammonium hydroxide in tetrahydrofuran at room temperature.

Unless indicated otherwise, the pressure of each of the above reactions is not critical. Generally, the reactions are conducted at a pressure of about one to about three atmospheres, preferably at ambient pressure (about one atmosphere).

The compounds of the formulas IIIa1-1 and IVa2-1 are capable of forming a wide variety of different salts with various inorganic and organic acids. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

Base salts may also be formed with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those that form non-toxic base salts with the herein described compounds of formula Ia-1. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

The compounds of the present invention are important in the manufacture of dihydroxyhexanoic acid derivatives of the formula Ia-1:

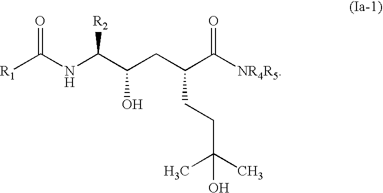

Compounds of the formula Ia-1 and their pharmaceutically acceptable forms (hereinafter also referred to, collectively, as “the active compounds”) are potent and selective inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment and prevention of the following disorders and conditions: autoimmune diseases (such as rheumatoid arthritis, Takayasu arthritis, psoriatic arthritis, juvenile arthritis, ankylosing spondylitis, type I diabetes (recent onset), lupus, inflammatory bowel disease, Chrohn's disease, optic neuritis, psoriasis, neuroimmunologic disease (multiple sclerosis (MS) primary progressive MS, secondary progressive MS, chronic progressive MS, progressive relapsing MS, relapsing remitting MS, worsening MS), polymyalgia rheumatica, uveitis, thyroiditis and vasculitis); fibrosis (such as pulmonary fibrosis (for example idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic inflammatory conditions including ocular inflammation, stenosis, lung inflammation (such as chronic bronchitis, chronic obstructive pulmonary disease, adult Respiratory Distress Syndrome, Respiratory Distress Syndrome of infancy, immune complex alveolitis), vascular inflammation resulting from tissue transplant or during restenosis (including, but not limited to, restenosis following angioplasty and/or stent insertion) and other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and chronic transplant rejection (including xeno-transplantation); HIV infectivity (co-receptor usage); granulomatous diseases (including sarcoidosis, leprosy and tuberculosis); Alzheimer's disease; chronic fatigue syndrome; pain; atherosclerosis; conditions associated with leptin production (such as obesity, cachexia, anorexia, type II diabetes, hyperlipidemia and hypergonadism); and sequelae associated with certain cancers such as multiple myeloma. This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the formula Ia-1 can be assessed according to procedures known to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

Chemotaxis Assay:

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists, were placed into the lower chambers of the Boyden chamber. A polycarbonate filter was then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotient is the % inhibition for the compound which can then be plotted using standard graphics techniques against the concentration of compound used. The 50% inhibition point is then determined using a line fit analysis for all concentrations tested. The line fit for all data points must have a coefficient of correlation (R squared) of >90% to be considered a valid assay.

The compounds of formula Ia-1 had IC₅₀ values of less than 25 μM, in the Chemotaxis assay.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery. Formulations of dihydroxyhexanoic acid derivatives are exemplified in co-pending U.S. patent application serial Nos. 60/300,255; 60/300,261; 60/300,256; and 60/300,260, all of which were filed on Jun. 22, 2001 and all of which are incorporated herein in their entireties for all purposes.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner. Moreover, quick dissolve tablets may be formulated for sublingual absorption.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch to provide for dry powder inhalation.

A proposed dose of the active compounds of the invention for oral, parenteral, nasal, or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents may be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, all of which are incorporated herein in their entireties for all purposes.

The compounds of the invention may also be utilized in combination therapy with other therapeutic agents such as those that inhibit immune cell activation and/or cytokine secretion or action (i.e. Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-Ig), BMS-224818 (CTLA4-Ig), RG-1046 (CTLA4-Ig), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, Anti-interleukin-6 receptor monoclonal antibody (MRA)), NSAIDS (aspirin, acetaminophen, naproxen, ibuprofen, ketoprofen, diclofenac and piroxicam), COX-2 inhibitors (Celecoxib, Valdecoxib, Rofecoxib, Parecoxib, Etoricoxib, L-745337, COX-189, BMS-347070, S-2474, JTE-522, CS-502, P-54, DFP), Glatiramer acetate, Interferon beta 1-a, Interferon beta 1-b, Mitoxantrone, Pimecrolimus, or agents that inhibit cell recruitment mechanisms (eg inhibitors of integrin upregulation or function) or alter leukocyte trafficking.

Experimental

The following examples are put forth so as to provide those of ordinary skill in the art with a complete disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. The following abbreviations are herein used:

AA is amino acid

AcOH is acetic acid

Boc is t-butoxy carbonyl

CDCl₃ is deuteriotrichloromethane

DMF is N,N-dimethylformamide

EtOAc is ethyl acetate

HCl is hydrochloric acid

HMDS is hexamethyldisilazane

IPE is isopropyl ether

MeOH is methanol

THF is tetrahydrofuran

g is grams

L is liter

M is molar

ml is milliliter

mmol is millimole

MHz is mega hertz

N is normal

psi is pounds per square inch

h is hours

min is minutes

sec is seconds

mp is melting point

RT is room temperature

Vacuo is in vacuum

˜is roughly approximate to*

HPLC is high pressure liquid chromatography

LCMS is liquid chromatograph mass spectrometer

NMR is nuclear magnetic resonance

TLC is thin layer chromatography

Note that all numbers provided herein are approximate, but effort have been made to ensure accuracy with respect to numbers (e.g., amounts, temperature, etc.); however some errors and deviations should be accounted for.

### EXAMPLE 1

### [2-(3-Fluoro-phenyl)-1-(5-oxo-tetrahydro-furan-2-yl)-ethyl]-carbamic acid tert-butyl Ester

Step 1:

2-tert-Butoxycarbonylamino-L-3-(3-fluorophenyl)-thioprocionic Acid S-propyl Ester

To a dry and nitrogen purged flask was added Boc-L-3-(fluorophenyl)alanine (18 g, 63.5 mmol). Anhydrous methylene chloride (180 mL) was added, and the solution was subsequently cooled to −10° C. Triethylamine (7.14 g, 69.9 mmol) was added over 10 min while keeping tempearature below −10° C. The mixture was stirred at −5 to −10° C. for 30 min, and isobutyl chloroformate (9.55 g, 69.9 mmol) was added via a drop funnel. After 30 min at −5° C., n-propyl mercaptan (5.32 g, 69.9 mmol) was added over 10 min via a drop funnel while keeping the temperature at −5° C. The mixture was allowed to warm to 0° C., and stirred for 30 min. HPLC assay of reaction aliquot showed complete conversion. 90 mL of water was added, and layers were separated. The methylene chloride layer was washed once more with water, dried over MgSO4, and concentrated. The residue was granulated in hexanes (50 mL). The mixture was filtered, and the filtrate was concentrated and filtered again to give a second crop. Both crops were of similar purity by HPLC assay. 21.3 g obtained. (98.2% yield). The material is an odorless and electrostatic crystalline solid.

HPLC: 99.7% pure by HPLC area (242 nm); Chiral assay not available due to lack of reference.

¹H NMR (400 MHz, CDCl₃) δ 0.92 (t, J=7.46 Hz, 3H), 1.38 (s, 9H), 1.560-1.60 (m, 2H), 2.82 (t, J=7.25 Hz, 2H), 2.95-3.13 (m, 2H), 4.54-4.62 (m, 1H), 4.94-4.99 (m, 1H), 6.83-7.25 (m, 4H); LC-MS: 364 (M+Na⁺), 242 (M+H⁺—Boc group).

Step 2

5-tert-Butoxycarbonylamino-6-L-(3-fluoro-phenyl)-4-oxo-hexanoic Acid Ethyl Ester

To a dry and nitrogen purged flask was charged 2-tert-Butoxycarbonylamino-L-3-(3-fluorophenyl)-thiopropionic acid S-propyl ester (10 mmol; 3.41 g), phthalic anhydride (3.00 eq; 30.0 mmol; 4.48 g), and dichlorobis(triethylphosphine) palladium (II) (0.05 eq; 0.499 mmol; 214 mg). 17 mL of anhydrous THF was added, followed by addition 3-ethoxy-3-oxopropylzinc bromide (6.00 eq; 60 mmol; 120 mL, 0.5 M solution in THF). After overnight stirring, the reaction was assayed by HPLC, and showed complete conversion. 1 N HCl (34 mL) and ethyl acetate (50 mL) were added, and the mixture was stirred for 5 min before layer separation. The organic phase was washed with saturated sodium bicarbonate and brine solution, then dried over MgSO₄. The solution was then concentrated under vacuum to give an oil residue. Upon standing, the residue solidified, and 5 mL of isopropyl ether was added. The mixture was granulated for 30 min at 0° C. and filtered. This gave 670 mg of desired product. The filtrate was added 3 mL of hexanes, cooled to −10° C., granulated for 30 min, and filtered. This gave 1.79 g of the product as second crop. The purity profiles of two crops were nearly identical. Combined yield 2.46 g (67.0%).

HPLC: 98.6% achiral; 100% chiral

NMR (400 MHz, CDCl₃) δ 1.24 (t, 7.0 Hz, 3H), 1.39 (s, 9H), 2.53-2.60 (m, 2H), 2.72-2.78 (m, 2H), 2.91 (dd, J=13.9 and 7.0 Hz, 1H), 3.17 (dd, J=5.8 and 13.9 Hz, 1H), 4.11 (q, J=7.0 Hz), 4.52 (dd, J=7.0 and 13.8 Hz), 5.08 (d, J=7.0 Hz), 6.87-7.27 (m, 4H). MS: 390 (M+Na⁺), 268 (M+H⁺—Boc group).

Step 3:

[2-(3-Fluoro-ohenyl)-1-(5-oxo-tetrahydro-furan-2-yl)-ethyl]-carbamic Acid Tert-butyl Ester

5-tert-Butoxycarbonylamino-6-L-(3-fluoro-phenyl)-4-oxo-hexanoic acid ethyl ester (1.0 g, 2.72 mml) was dissolved in THF (10 ml) and the solution was cooled to −78° C. 1.0 M solution of N-selectride in THF was added dropwise while keeping reaction below −72° C. over 30 min. After 10 min stirring at −78° C., HPLC assay showed reaction completion. Acetic acid (0.62 ml) in THF (10 ml) was added dropwise. The reaction was allowed to warm to room temperature and subsequently heated at reflux for 10 min. After cooling to room temperature, ethyl acetate (10 ml) and 1 N HCl (10 ml)) were added. The layers were separated, the organic layer was washed with saturated sodium bicarbonate and brine. After drying over MgSO₄, the organic phase was concentrated to an oil in vacuo. The resulting residue was granulated in IPE (2 mL), then hexanes (2 mL) was added. The mixture was granulated at ambient temperature for 1 h. The mixture was filtered and rinsed with IPE/hexanes (1:1). This gave the desired product as a white solid (673 mg, 76.5%).

HPLC: Chiral 97.8% (enantiomer 1.1%, diastereomer 1.0%); Achiral 98.0% Identical with authentic sample.

### EXAMPLE 2

### [2-(3-Fluoro-phenyl)-1-(5-oxo-tetrahydro-furan-2-yl)-ethyl]-carbamic Acid tert-butyl Ester

Step 2 of Example 1 was Replaced with the Following Procedure for Producing 5-tert-Butoxycarbonylamino-6-L-(3-fluoro-phenyl)-4-oxo-hexanoic Acid Ethyl Ester

To a dry and nitrogen purged flask was charged 0.5 M ZnCl2 solution in THF (4 eq, 8 mmol, 16 mL). [1-(ethoxycyclopropyl)oxy]trimethylsilane (8 eq, 16 mmol, 2.81 g) was added via a syringe. The mixture was stirred at ambient temperature for 4 h, then stripped to an oil under vacuum at 25-30° C. THF (10 mL) was added. This solution was transferred to another flask that contained CP-939468 (682 mg, 2.0 mmol), phthalic anhydride (3.00 eq; 6.0 mmol; 897 mg) and dichlorobis-(triethylphosphine)palladium (II) (0.05 eq; 0.1 mmol; 42.8 mg) in DMAc (3.4 mL). After overnight stirring at ambient temperature, the reaction was assayed by HPLC, and showed desired product, but the reaction was incomplete (28% starting material by HPLC area). The reaction was worked up as described earlier. The final concentrated residue was passed through a silica gel pad eluting with 20% ethyl acetate/hexanes. The product containing fractions were combined and concentrated to give an oil, after standing overnight, the oil solidified. The material was triturated in hexanes and filtered to give 285 mg product. (54% yield after excluding starting material).

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of making a compound of the formula (V-1): 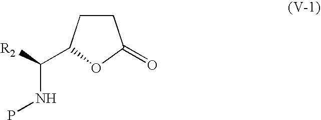
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-H N(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises:
a) reacting a compound of the formula (VIh-1) 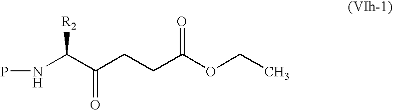
with a reducing agent, and
c) cyclizing the compound so formed with heat and an acid catalyst.

2. The method of claim 1, further comprising formation of the compound of the formula (VIh-1) by reacting a compound of the formula (VIIa-1) 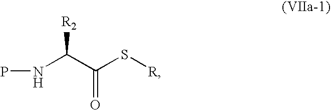
wherein R is a (C₁-C₂₀)alkyl, (C₃-C₁₀)cycloalkyl, aryl, or (C₂-C₉)heteroaryl with an organozinc compound in the presence of a palladium catalyst and a racemization scavenger.

3. The method of claim 2, further comprising formation of the compound of the formula (VIIa-1) by reacting a compound of the formula (VIII-1) 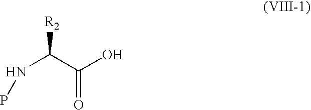
with a compound of the formula HS-R.

4. A method of making a compound of the formula (V-1): 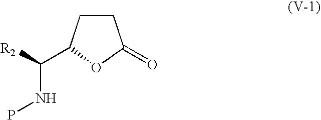
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-H N(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises:
a) reacting a compound of the formula (VIII-1) 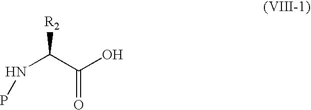
with a compound of the formula HS-R, wherein R is a (C₁-C₂₀)alkyl, (C₃-C₁₀)cycloalkyl, aryl, or (C₂-C₉)heteroaryl, to form a compound of the formula (VIIa-1) 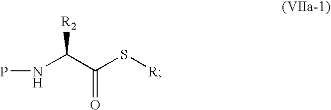
b) reacting the compound so formed with an organozinc compound in the presence of a palladium catalyst and a racemization scavenger to form a compound of the formula (VIh-1) 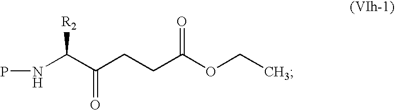
c) reacting the compound so formed with a reducing agent; and
e) cyclizing the compound so formed with heat and an acid catalyst.

5. The method of claim 2, wherein with the organozinc compound has the formula formula X—Zn—(C₁-C₆)alkyl-(C═O)—O—R, wherein X is a halogen and R is a (C₁-C₂₀)alkyl, (C₃-C₁₀)cycloalkyl, aryl, or (C₂-C₉)heteroaryl. 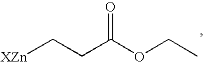
wherein X is a halogen.

6. The method of claim 4, wherein with the organozinc compound has the formula formula X—Zn—(C₁-C₆)alkyl-(C═O)—O—R, wherein X is a halogen and R is a (C₁-C₂₀)alkyl, (C₃-C₁₀)cycloalkyl, aryl, or (C₂-C₉)heteroaryl. 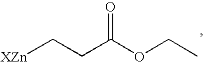
wherein X is a halogen.

7. The method of claim 2, wherein with the organozinc compound has the formula 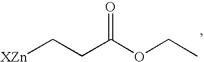
wherein X is a halogen.

8. The method of claim 4, wherein with the organozinc compound has the formula 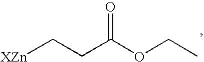
wherein X is a halogen.

9. The method of claim 2, wherein the organozinc compound has the formula

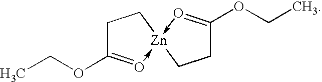

10. The method of claim 4, wherein the organozinc compound has the formula

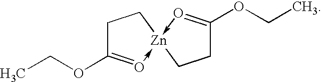

11. The method of claim 2, wherein the racemization scavenger is a carboxylic acid anhydride or a carboxylic acid ester.

12. The method of claim 4, wherein the racemization scavenger is a carboxylic acid anhydride or a carboxylic acid ester.

13. The method of claim 2, wherein the racemization scavenger has the formula aryl-(C═O)—O—(C═O)-aryl, R—(C═O)—O-aryl, or aryl-(C═O)—O-aryl.

14. The method of claim 4, wherein the racemization scavenger has the formula aryl-(C═O)—O—(C═O)-aryl, R—(C═O)—O-aryl, or aryl-(C═O)—O-aryl.

15. The method of claim 13, wherein the racemization scavenger is phthalic anhydride, 4-nitrophenyl acetate, or 4-fluorophenyl acetate.

16. The method of claim 14, wherein the racemization scavenger is phthalic anhydride, 4-nitrophenyl acetate, or 4-fluorophenyl acetate.

17. The method of claim 1, wherein P is carbobenzyloxy, t-butoxy carbonyl or 9-fluorenyl-methylenoxycarbonyl.

18. The method of claim 4, wherein P is carbobenzyloxy, t-butoxy carbonyl or 9-fluorenyl-methylenoxycarbonyl.

19. The method of claim 1, wherein P is t-butoxy carbonyl.

20. The method of claim 4, wherein P is t-butoxy carbonyl.

21. The method of claim 1, wherein R₂ is phenyl-(CH₂)ₘ— or (C₂-C₉)heteroaryl-(CH₂)ₘ—, wherein each of said phenyl or (C₂-C₉)heteroaryl moieties may be optionally substituted with one, two, or three substituents independently selected from the group consisting of hydrogen, halogen, CN, (C₁-C₆)alkyl, or hydroxy.

22. The method of claim 4, wherein R₂ is phenyl-(CH₂)ₘ— or (C₂-C₉)heteroaryl-(CH₂)ₘ—, wherein each of said phenyl or (C₂-C₉)heteroaryl moieties may be optionally substituted with one, two, or three substituents independently selected from the group consisting of hydrogen, halogen, CN, (C₁-C₆)alkyl, or hydroxy.

23. The method of claim 1, wherein R₂ is 3-fluoro-benzyl.

24. The method of claim 4, wherein R₂ is 3-fluoro-benzyl.

25. The method of claim 2, wherein R is a (C₁-C₆)alkyl, aryl, or (C₂-C₉)heteroaryl.

26. The method of claim 4, wherein R is a (C₁-C₆)alkyl, aryl, or (C₂-C₉)heteroaryl.

27. A compound of the formula (VIh-1) 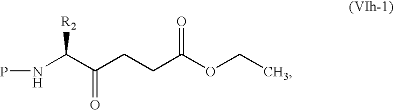
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4.

28. A compound of the formula (VIIa-1) 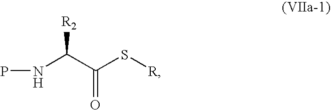
wherein:
P is a protecting group;
R is a (C₁-C₂₀)alkyl, (C₃-C₁₀)cycloalkyl, aryl, or (C₂-C₉)heteroaryl;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4.

29. The compound of claim 27, wherein P is carbobenzyloxy, t-butoxy carbonyl or 9-fluorenyl-methylenoxycarbonyl.

30. The compound of claim 28, wherein P is carbobenzyloxy, t-butoxy carbonyl or 9-fluorenyl-methylenoxycarbonyl.

31. The compound of claim 29, wherein P is t-butoxy carbonyl.

32. The compound of claim 30, wherein P is t-butoxy carbonyl.

33. The compound of claim 27, wherein R₂ is phenyl-(CH₂)ₘ— or (C₂-C₉)heteroaryl-(CH₂)ₘ—, wherein each of said phenyl or (C₂-C₉)heteroaryl moieties may be optionally substituted with one, two, or three substituents independently selected from the group consisting of hydrogen, halogen, CN, (C₁-C₆)alkyl, or hydroxy.

34. The compound of claim 28, wherein R₂ is phenyl-(CH₂)ₘ— or (C₂-C₉)heteroaryl-(CH₂)ₘ—, wherein each of said phenyl or (C₂-C₉)heteroaryl moieties may be optionally substituted with one, two, or three substituents independently selected from the group consisting of hydrogen, halogen, CN, (C₁-C₆)alkyl, or hydroxy.

35. The compound of claim 33, wherein R₂ is 3-fluoro-benzyl.

36. The compound of claim 34, wherein R₂ is 3-fluoro-benzyl.
